(19) United States
(12) Patent Application Publication
(10) Pub. No.: US 2009/0247633 A1
(43) Pub. Date: Oct. 1, 2009

(54) NUTRIENT EXTRACTS DERIVED FROM GREEN PLANT MATERIALS

(75) Inventor: Lance B. Crombie, Northfield, MN (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: Pandora Select Partners L.P. and Whitebox Hedge High Yield Partners, L.P., Minneapolis, MN (US)

(21) Appl. No.: 12/092,665

(22) PCT Filed: Nov. 3, 2006
(86) PCT No.: PCT/US06/60541

§ 371 Date: Oct. 13, 2008

(30) Foreign Application Priority Data
Nov. 4, 2005 (US) 67733465

(51) Int. Cl. A61K 31/202; A61K 31/20; A61K 31/201; A61P 9/10

(52) U.S. Cl. 514/560; 514/558

(57) ABSTRACT

An extract from green plant materials includes controlled concentrations of nutritional components. In one embodiment, the nutritional components include at least one xanthophyll, at least one hydrocarbon carotene, and at least one fatty acid wherein the xanthophyll, hydrocarbon carotene, and fatty acid each have about equal weight percentages. In a one embodiment, the green plant is alfalfa, the xanthophyll is lutein, the hydrocarbon carotene is β-carotene, and the fatty acids include an omega-3 essential fatty acid, an omega-6 essential fatty acid, or a combination of the acids thereof.

### BACKGROUND OF THE INVENTION

Nutritional supplements have become an increasingly popular source to obtain nutrients that are not produced naturally by the body, and/or are not being obtained in sufficient quantities through diet. Many nutritional supplements combine multiple nutrients into a single supplement to purportedly provide a wide range of health benefits.

One class of nutrients contained in certain supplements are carotenoids, which may be derived from a variety of natural sources. Carotenoids may be classified as hydrocarbon carotenes, or as xanthophylls, which are oxygenated derivatives of carotenes. Carotenoids have been shown to have antioxidant properties and have been studied for the prevention of cancer and other human diseases. Representative examples of carotenes include α-carotene, β-carotene, and lycopene. Xanthophylls are antioxidants and can contribute to eye health. Examples of xanthophylls include lutein, astaxanthin, canthaxanthin, zeaxanthin, cryptoxanthin, and capsorubin.

Carotenoids are naturally present in edible leaves, flowers, and fruits, and are readily obtained from flowers (e.g., marigold), berries, and root tissue (e.g., carrots). Hydrocarbon carotenes, such as β-carotene and lycopene, are typically present in an uncombined free form, which is entrapped within chloroplast bodies within plant cells. Xanthophylls, such as lutein, are abundant in a number of yellow or orange fruits and vegetables such as peaches, mango, papaya, prunes, acorn squash, and oranges. Some xanthophylls are present in plant flowers, such as marigolds, as long-chain fatty esters, typically diesters of acids such as palmitic and myristic acids. Generally, the free forms of carotenoids are present in the chloroplasts of green plants such as alfalfa, spinach, kale, and leafy green plant materials. The free form of the carotenoids provides better adsorption when consumed in foods or as a supplement.

Lutein is a xanthophyll found in high concentrations in the macula of the eye and in the central part of the retina. It serves important roles in vision to help filter ultraviolet wavelengths of light to prevent damage to the eye lens and macula. Lutein's antioxidant properties are believed to help protect the macula, which is rich in polyunsaturated fats, from light-induced free radicals. Lutein cannot be produced by the body and consequently, must be ingested. Thus, lutein has become increasingly used in nutritional supplements for the prevention and/or treatment of vision losses due to macular degeneration, cataracts, and retinitis pigmentosa.

The most common source of extracted lutein is from marigold flower petals, which contain one of the highest levels of lutein known and have a low concentration of other carotenoids. Methods of the purification of lutein-fatty acid esters from marigold flower petals are reported in U.S. Pat. Nos. 4,048,203; 5,382,714; and 5,648,564, in which dried, ground marigold flower petals are extracted with a hydrocarbon solvent.

However, extraction of lutein from green plants may be beneficial because it removes the need for the additional chemical step of saponification or ester cleavage to release the free lutein, which is the desired form for best absorption as consumed. However, the extraction and purification of lutein, carotenes, and fatty acids from plants has not been economical in the past because many expensive and time-consuming purification steps have been required to separate them from the large quantities of other compounds present in the plant materials.

Lutein is also abundantly present in a free, non-esterified form in green plants such as alfalfa, broccoli, green beans, green peas, lima beans, cabbage, kale, spinach, collards, mustard greens, turnip greens, kiwi, and honeydew. Green plants may also be rich in a variety of additional nutrients. For example, alfalfa is rich in proteins, minerals, and vitamins. It contains all 21 amino acids, and has significant concentrations of vitamins A, D, E, B-6, and K, calcium, magnesium, chlorophyll, phytoestrogens, phosphorous, iron, potassium, trace minerals and several digestive enzymes. It contains also several saponins, many sterols, flavonoids, coumarins, alkaloids, acids, additional vitamins, amino acids, natural sugars, proteins (25% by weight), minerals, trace elements, and other essential nutrients.

Another popular nutrient used in nutritional supplements are fatty acids, which have been shown beneficial to health in a variety of ways, including cancer treatment, cardiovascular health, and as an anti-inflammatory substance. In particular, essential fatty acids (EFAs) are recognized as being an important nutrient, which cannot be made by the human body and must instead be ingested from external sources. Examples of EFAs include linolenic, linoleic, and oleic acids, and they are found in natural sources. In addition to green plant materials such as alfalfa, additional sources of various essential fatty acids include flax seeds and oil, walnuts, fish oil, canola, borage oil, evening primrose oil, black current oil, vegetable oils, eggs, poultry, red meat, animal and vegetable fats, and olive oil.

Fatty acids are aliphatic hydrocarbon chains, typically of 12 to 22 carbon atoms, having a carboxylic acid group (COOH) at one end, usually referred to as the alpha (α) end. The chain end opposite the α end is called the omega (ω) end. A number following omega indicates where a carbon-carbon double bond is. For example, omega-3 indicates a carbon-carbon double bond in the third carbon-carbon bond from the ω end of the chain, and omega-6 indicates a carbon-carbon double bond in the sixth carbon-carbon bond from the ω end of the chain. A monounsaturated fatty acid would have a single carbon-carbon double bond in the chain, while a polyunsaturated fatty acid would have at least two carbon-carbon double bonds. A saturated fatty acid has no carbon-carbon double bonds.

Omega-3 fatty acid is a polyunsaturated, essential fatty acid having at least two and a maximum of 6 carbon-carbon double bonds in a carbon chain ranging from 18 to 22 carbon atoms. Some common fatty acids are known by two names. For example, α-linolenic acid (ALA), having 18 carbon atoms and three double bonds, is known also as omega-3 fatty acid, the name difference being due to which end of the chain is the reference point. Other common omega-3 fatty acids are docosapentaenoic acid (DHA), having 22 carbon atoms and six double bonds and eicosapentaenoic acid (EPA), having 20 carbon atoms and five double bonds. The human body converts ALA into DHA and EPA, which are more readily used.

Omega-6 fatty acid is a polyunsaturated, essential fatty acid having at least two carbon-carbon double bonds in a carbon chain ranging from 18 to 22 carbon atoms, with one carbon-carbon double bond at the sixth carbon from the ω end. Examples of omega-6 fatty acids are γ-linolenic acid (GLA), having 18 carbon atoms and three carbon-carbon double bonds; linoleic acid, having 18 carbon atoms and two carbon-carbon double bonds; and arachidonic acid, having 20 carbon atoms and four carbon-carbon double bonds. There are numerous positional isomers of linoleic acid having conjugated double bonds, commonly known as CLAs. Two common CLA isomers are known as the cis-9, trans-11 and cis-10, trans-12 isomers. A common omega-9 fatty acid is oleic acid, a mono-unsaturated fatty acid. Palmitic acid, also known as n-hexadecanoic acid and hexadecyclic acid, is a 16-carbon, saturated fatty acid.

It has been reported that because omega-6 EFAs can inhibit the absorption of omega-3 EFAs, their combined ingestion must be balanced to avoid adverse effects. It has been reported that the ratio of ingested omega-6 to omega-3 should not exceed about 4-5 to 1, that it should preferably be 1-2 to 1, or even less than 1, and that western diets can have such a ratio of 10-30 to 1 and be a cause of health problems. It may be advantageous to form a nutritional supplement in which the omega-3 is greater than the omega-6.

Although various combinations and concentrations of the above-described nutrients may be formed into a single supplement, a potential drawback to such supplements is that the nutrients would necessarily be derived from multiple raw material sources. This may cause several problems, including manufacturing inefficiencies and other drawbacks. One drawback in particular is that extracts obtained from multiple natural sources may produce varying concentrations of the essential nutrients. The purity and efficacy of such separately obtain nutritional components should be known before being combined into a nutritional supplement. therefore, it would be beneficial to form such nutritional supplements from an extract derived from single natural source material, in which the extract has consistent predictable concentrations of desired essential nutrients even though the natural source may contain varying concentrations of the essential nutrients.

### SUMMARY OF THE INVENTION

The present invention is directed to an extract of green plant materials that contains controlled concentrations of desired nutritional components. In one embodiment, the desired extract may particularly include at least one xanthophyll such as lutein, a hydrocarbon carotene such as β-carotene, and at least one fatty acid in substantially similar concentrations. For example, the extract may include between about 30-40 wt % of each of these nutritional components. This extract may be combined with a suitable nutritional carrier, such as cyclodextran, to form a nutritional supplement.

In another embodiment, the present invention is directed to an extract of green plant material in which the extract includes controlled concentrations of at least one xanthophyll such as lutein, at least one hydrocarboncarotene such as β-carotene, and at least one fatty acid. In one embodiment, the green plant materials consist of a single source of green plant material such as alfalfa.

The concentrations of the extracted nutritional components may be controlled by a supercritical extraction process that is performed at particular pressures, temperatures and/or volumes for particular amounts of time. For example, a supercritical extraction process may be performed in two phases. In a particular embodiment, the green plant material may be subjected to a two-part supercritical extraction process in which the green plant material is first brought to a pressure of about 15 MPa and temperature of about 25° C. for about 10-15 minutes in the presence of a suitable supercritical fluid to form a first extract. A second extraction process is then performed on the remaining green plant material in which the pressure is increased to about 40 MPa, while maintaining a temperature of about 25° C., for about 60 minutes in the presence of a supercritical fluid to form a second extract. The first extract may be discarded. The pressure of the second extract may then be lowered so that the extract with the controlled concentrations of the at least one xanthopyll, the at least one hydrocarbon carotene, and the at least one fatty acid may be collected.

In another embodiment, the present invention provides an extract of green plant materials which includes at least one xanthophyll, at least one hydrocarbon carotene, and at least one fatty acid, each having a concentration of between about 20-40 wt %. In a particular embodiment, the xanthophyll is lutein, the hydrocarbon carotene is a β-carotene, and the fatty acid is a combination of α-linolenic acid and linoleic acid. In this particular embodiment, the ratio of concentration of α-linolenic acid to linoleic acid in weight percents may be between about 3:1 and 1:1. In a more particular embodiment, the ratio of concentration of α-linolenic acid to linoleic acid in weight percents may be about 2:1.

In still another embodiment, the present invention provides an extract of green plan materials which includes at least one xanthophyll, at least one hydrocarbon carotene, and at least one fatty acid, each having about equal concentrations. In a particular embodiment, the xanthophyll is lutein, the carotene is a β-carotene, and the fatty acid is a combination of Omega-3 and Omega-6 fatty acids in a ratio of between about 3:1 and 1:1 omega-3:omega 6 fatty acids.

In yet another embodiment, the present invention provides a nutritional composition which includes a carrier and an extract of green plant material where the extract includes controlled concentrations of at least one xanthophyll, at least one hydrocarbon carotene such as β-carotene, and at least one fatty acid. In a yet another embodiment, the carrier is cyclodextran. In still another embodiment the cyclodextran forms between about 60-95 wt % of the composition, and the lutein, the hydrocarbon carotene, and the fatty acid of the extract each form between about 1-15 wt % of the composition. The extract may include about equal concentrations of lutein, hydrocarbon carotene and at least one fatty acid. This composition may be used as a nutritional supplement.

In another embodiment, the present invention provides a composition which includes a carrier and an extract from green plant material where the extract includes at least one xanthophyll such as lutein, a carotene such as β-carotene, and at least two fatty acids. In this embodiment, the fatty acids include at least one omega-3 essential fatty acid and at least one omega-6 essential fatty acid. In a yet another embodiment, at least one omega-3 essential fatty acid is α-linolenic acid and the at least one omega-6 essential fatty acid is linoleic acid. In this embodiment, the composition may have reduced, or may be free of, additional fatty acids that were present in the green plant material.

In still another embodiment, the present invention provides an extract formed by the process of performing a first supercritical fluid extraction of a green plant material at a first pressure and temperature to obtain a first extract, performing a second supercritical fluid extraction of the green plant material at a second pressure and temperature to obtain a second extract that includes a higher concentration of lutein than the concentration of lutein in the first extract, and separating the second extract from the supercritical fluid.

In yet another embodiment, the present invention provides an extract formed by the process of performing a first supercritical fluid extraction of a green plant material at a first pressure and temperature to obtain a first extract, performing at least one additional supercritical fluid extraction of the green plant material at least one additional pressure and temperature to obtain at least one additional extract, wherein at least one of the additional extracts includes a higher concentration of lutein than in the first extract, and separating at least one of the additional extracts from the supercritical fluid.

### BRIEF DESCRIPTION OF THE DRAWINGS

FIG. 1 illustrates a flow-chart for the fractionation and extraction of lutein according to an embodiment of the present invention.

FIG. 2 illustrates a lutein extraction chamber and a process flow diagram with collection system according to an embodiment of the present invention.

### DETAILED DESCRIPTION

A variety of green plant materials may be used as the starting source material for the present invention. Suitable green plant materials may include alfalfa, wheat grass, barley grass, broccoli, kale, spinach, cabbage, soybeans, green beans, mustard greens, turnip greens, collards, and green peas. In one embodiment, alfalfa is the green plant source material.

In one embodiment, one or more extracts are obtained by supercritical fluid extraction using a supercritical fluid (SCF) such as carbon dioxide. The process parameters, including the pressure, temperature, volume and/or extraction time of the supercritical extraction system are controlled during extraction to obtain a controlled concentration of lutein, β-carotene, and fatty acids within the capability of the process and materials used. In another embodiment, multiple supercritical extractions may be performed to obtain multiple extracts having differing types and concentrations of nutrients.

Although the starting green plant material may be utilized in any form (e.g. wet or dry) that includes and preserves the desired nutrients for supercritical extraction, a wet or dried chloroplast-rich fraction of a green plant may be particularly useful for enhanced extraction of carotenoids. The chloroplast-rich fraction may be separated from other plant fractions by a process that includes the use of heat, acids, centrifugation, electrical fields, or flocculants. The chloroplast-rich fraction may be dried to 5-50% moisture with hot air, infrared heat, microwave radiation or a vacuum oven prior to the extraction with super critical fluid to preserve the desired components. Prior to supercritical fluid extraction, the chloroplast rich fraction may be washed with an aqueous solution. This washing step may remove bitter flavors from the chloroplast rich fraction to provide a more palatable fraction for use in a nutritional supplement. Alternatively, the starting material may be dried in such a manner that it preserves the desired nutrient(s) for subsequent supercritical extraction. Additionally, in this embodiment, the green plant material may be dried in the absence of oxygen if the desired nutrient is sensitive to oxidation by air or oxygen.

SCFs, which are gases above their critical pressure and temperature, have been used in certain industries to perform extractions. SCFs are dense gasses in a separate phase, which is distinct from normal gas phase. SCFs have a density and solvating power similar to that of a liquid and diffusion rates similar to that of a gas. Supercritical fluids are unlike liquids because their solvent power is highly sensitive to pressure changes and may be varied over wide limits by changing the pressure.

SCF extraction offers a relatively rapid, simple and inexpensive technique to perform purification or compound preparations. Most compounds, once dissolved, can quickly and cleanly be precipitated or removed from the supercritical fluids by lowering the pressure or temperature or both to achieve separation. Because a slight change in the pressure or temperature of a system causes significant change in solubility, the use of SCF enables a highly efficient isolation procedure of the desired components to be extracted. Using the method of post-extraction fractionation with a column designed to allow for temperature and pressure drops at different levels to gain the desired results may effect further concentration and purification.

Although generally SCF extraction is performed in batches, the present invention may also be formed using a continuous method for obtaining a plurality of extracts of the invention from green plant material. A plurality of supercritical extractions may be performed at a plurality of pressures to obtain a plurality of extracts. For example, one of the extracts may contain substantial amounts of lutein. Another extract may contain substantial amounts of hydrocarbon carotene. Other extracts may contain fatty acids, xanthophylls, zeaxanthin, astaxanthin, canthaxanthin, capsorubin and cryptoxanthin. Such extracts may be obtained by optimizing the pressure and temperature environment at which the extract is obtained to provide an extract having a substantial concentration of the desired substance.

In yet another embodiment of the present invention, a lutein-enriched extract is obtained from a first supercritical extraction performed at a first pressure and temperature to obtain a first extract. A second supercritical extraction is then performed at a second pressure and temperature to obtain a second extract. The second extract may have a higher concentration of lutein than the first extract and may include controlled concentrations of β-carotene and fatty acids in weight percent about equal to the concentration of lutein. Alternatively, the concentrations of β-carotene and fatty acids in weight percent may be less than the concentration of lutein.

To form the controlled concentrations of the nutritional components in the extract, a first extraction is performed at a first temperature and pressured to obtain a first extract, and a second extraction is performed at a second temperature and pressure to obtain a second extract. By optimizing the temperature and pressure at which the first and second supercritical extractions are performed, each extract may contain a controlled concentration of a particular substance, such as a desired hydrocarbon carotene, xanthophylls and/or fatty acid. In one embodiment, the first extraction is carried out to remove certain types and amounts of nutrients and/or other materials such that the second extract contains the desired types of nutritional components at controlled concentrations.

After performing the second supercritical extraction, the desired extract may be separated from the second supercritical fluid by lowering the pressure of the second supercritical extract such that the lutein precipitates out of the second supercritical extract and onto a desired carrier. The first extraction may be separated in a similar manner. In one embodiment, the pressure of the first supercritical extract may be lowered to about 10 MPa and the pressure of the second extract may be lowered to about 30 MPa. The first and/or second extract may then be processed to form an end product suitable for consumption.

In one particular embodiment, a first extraction is performed at a first temperature and pressure to obtain a first extract, and a second extraction is performed at a second temperature and pressure to obtain a second extract. During the first and second supercritical extractions of the green plant material, the first and second pressures may be between about 8 MPa to about 200 MPa, more particularly between about 10 MPa to about 120 MPa. In one embodiment, the first pressure is lower than the second pressure. For example, the first pressure may be between about 10 and about 40 MPa, and the second pressure may be between about 41 and about 80 MPa. Alternatively, the first pressure may be about 20 MPa and the second pressure may be about 65 MPa. The temperature during the supercritical extractions may be between about 31° C. to about 150° C., or between about 31° C. to about 40° C., or about 35° C. The temperature may be varied or remain constant during the extractions. For example, dried fresh cut alfalfa may yield about 1 gram of lutein per pound of dried raw alfalfa, about 1 gram of β-carotene per pound of dried raw alfalfa, and about 2 grams of omega-3 fatty acid per pound of dried raw alfalfa. The yields from other fresh crops of green plants may vary.

In another embodiment, a green plant material is subjected to a first supercritical extraction at a pressure of 15 MPa and a temperature of about 25° C. for about 10-15 minutes to form a first extract. The green plant material is then subjected to a second supercritical extraction at a pressure of 15 MPa, and at a temperature of about 25° C. for about 60 minutes to form a second extract. The second extract is then precipitated out of the supercritical fluid as described above to form an extract with substantially similar concentrations of lutein, β-carotene, and fatty acid. For example, the lutein, β-carotene, and fatty acid may each make up about between about 25 wt % and about 40 wt %, or between about 30 wt % and about 35 wt %, or 33 wt % of the extract.

In another embodiment the fatty acid component is made up by about 65 wt % of α-linolenic acid, about 30 wt % of linoleic acid, and about 5 wt % of other fatty acids. In this embodiment, the supercritical extractions may serve to concentrate essential omega-3 and omega-6 fatty acids by removing the excess fatty acids that are present in the raw green plant material. For example, the fatty acid content of raw alfalfa may contain about 22% saturated fatty acids, about 3% monounsaturated fatty acids, and about 75% polyunsaturated fatty acids. The amount of linolenic acid, an omega-3 fatty acid, may be 51% of the total fatty acid content while the amount of linoleic acid, an omega-6 fatty acid, may be about 23% of the total fatty acid content. The extract derived from the raw alfalfa with the supercritical fluid extraction process described above may result in an increase in the linolenic acid to about 65% of the total fatty acid content of the extract, and an increase in the linoleic acid content to about 30% of the total fatty acid content of the extract, with other fatty acids making up the remaining 5% of the total fatty acid content.

The steps of processing raw green plant material into an extract with controlled concentrations of nutritional components are described below.

### Pre-Extraction Processing of Green Plant Material

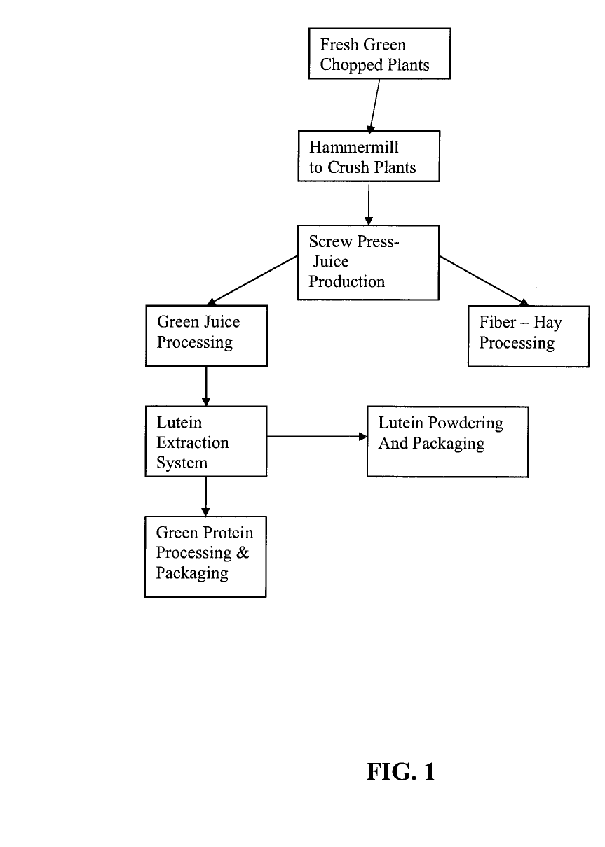

The green plant material may be processed in a variety of ways prior to performing supercritical extraction to obtain a desired starting material. In one embodiment, the green plant material is subjected to a wet fractionation process, e.g., as illustrated in FIG. 1 of U.S. Pat. Nos. 6,737,552 or 6,909,021, incorporated herein by reference. For this embodiment, pre-bloom alfalfa may be harvested with standard farm equipment and then cut or chopped into ½ to 4-inch lengths. This cutting or chopping process is generally performed within 1 hour after harvesting to preserve the desired extractable nutritional components. The cut or chopped alfalfa may then be crushed or macerated with rollers or with hammermill devices that rupture plant cell walls. The macerated green crop may then be squeezed in an appropriate pressing device, screw press, or other press separate the green plant juices from the fibrous plant material.

The residue fibrous plant fraction, or wet fiber fraction of alfalfa, typically possesses 55-65% moisture, 14-18% protein, and has most of the typical nutritional value of green forages. This fraction may be used for ruminant feed for beef or dairy cows in either wet or dry form.

The green plant juice is a mixture of cell sap materials, which include water, salts, chloroplasts, and cytoplasmic proteins, enzymes and cell compounds. The juice may be further treated by one of several methods to separate desired components. In one method, the juice is typically subjected to heat coagulation at 60° C. for the chloroplast fraction and at 85° C. for the cytoplasmic fraction. Alternatively, the juice may be treated by acid precipitation, by density separations in centrifugal fields, or by direct current electrical fields. These techniques produce three general fractions: (a) a green protein chloroplast fraction; (b) a white cytoplasmic protein fraction; and (c) a brown juice fraction. In another extraction method, separation of the green protein concentrates from the brown juice is performed by centrifugation or filtration methods.

In one embodiment, the green protein chloroplast fraction of alfalfa is the starting green plant material for the supercritical extraction. This fraction is rich in plant chloroplasts and is typically composed of 50-55% protein on a dry weight basis and has 1.8 to 3.5 g xanthophylls per kg. The green chloroplast fraction may be used wet or may be dried prior to extraction of carotenoids. The dried form may produce a more stable material for extraction.

The fractions of the green plant juice may be dried under gentle conditions to preserve the desired components. Drying may be accomplished with steam heated air or other hot inert gases, infrared heat, microwave, vacuum oven devices, or any other method or combination of methods to remove water to the desired level.

Washing the green protein chloroplast fraction with an aqueous solution or water just prior to supercritical extraction may be advantageous. This washing process may remove off-flavors and bitter grassy flavors from the protein concentrate fraction and may make the extract more palatable for subsequent human consumption. Lutein has very little solubility in water, so the water wash causes only minor loss of product. This washing step may be particularly beneficial if the post-extraction green protein chloroplast fraction is used as part of a nutritional supplement.

Although alfalfa is used as the green plant material in the reported embodiment, any fresh green plant material that can be processed by wet fractionation may be used, including wheat grass, barley grass, broccoli, kale, spinach, cabbage, soybeans, green beans, mustard greens, turnip greens, collards, or green peas. For example, the wet fractionation process reported above may be easily adapted to wheat grass and barley grass. Since the wet fractionation is similar for alfalfa and grasses, the process is the same for most fresh green plants.

### Supercritical Extraction Process

Once a suitable green plant material is obtained, supercritical extraction may be performed by passing SCFs through the green plant material. The SCF used in the method of the present invention may include CO₂, CH₂CH₂, CH₃CH₃, N₂O or other suitable supercritical fluids. A co-solvent may be used along with the supercritical fluid to increase the solvation power for polar analytes that do not readily dissolve in supercritical fluids. Co-solvents are often referred to as entrainers or modifiers, and are typically a liquid organic solvent such as methanol, ethanol, propylene carbonate, acetone, tetrahydrofuran, formic acid, propylene glycol, or ethyl acetate that are blended with the carbon dioxide. With an entrainer, the solvent system has a much higher polarity and is able to solubilize more polar analytes for extraction. Entrainers have been shown to substantially increase the solubility of zeaxanthin in supercritical carbon dioxide as reported, in part, in U.S. Pat. No. 5,747,544. In obtaining one embodiment, the SCF includes ethanol as an entrainer at 1-5% concentration in the extracted material. This entrainer might produce a better extraction at lower pressures and produce a more suitable end product for conversion into a dry powder with cyclodextrans or other insolubilizing materials.

In one embodiment of the invention, the SCF is carbon dioxide, which has a critical pressure of 1,070 psi (about 7.4 MPa) and a critical temperature of 31° C. Solvation power increases as pressure and temperature is raised above the critical pressure and temperature. Supercritical CO₂ may be manipulated at room temperature, making the handling of heat-vulnerable substances easy and safe. Fire and explosion hazards associated with large-scale extractions using organic solvents are eliminated with this solvent.

In practice, the SCF is passed through the green plant materials inside an extraction vessel. The SCF diffuses into the pores of the green plant material matrix, solubilizes the nutritional components (e.g., lutein, β-carotene, and fatty acids) of interest, and then carries the extract containing the nutritional components away from the green plant matrix in a solution. The solubilized nutritional components are then collected, and the green plant matrix (now without the extract) is left behind in the extraction vessel. Changing SCF pressure or temperature may control solvent strength in a precisely controlled manner. SCFs have favorable diffusion and viscosity coefficients providing for good mass transfer characteristics. As opposed to conventional solvent extraction, any residual CO₂ left in the extract after separation is inert and non-toxic, such that human consumption of the material is not harmful.

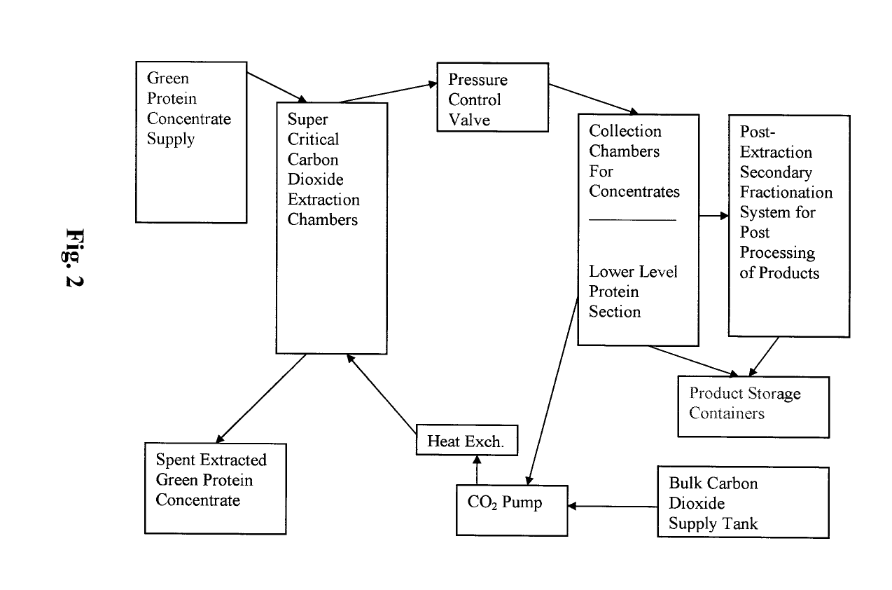

A SCF extraction process, e.g., as illustrated in FIG. 2 of U.S. Pat. Nos. 6,737,552 or 6,909,021, is performed in, for example, a round thick-walled, very-high-pressure chamber, engineered to withstand pressures up to about 120 MPa (1,450-17,400 psi), more particularly up to about 70 MPa (10,150 psi). The chamber has openings for adding a suitable charge of green plant protein concentrate at the top and for removal of the charge after extraction at the bottom, for example, by the use of a suitable double valve. Appropriate pipes and pump systems direct the supercritical carbon dioxide fluid into the bottom of the chamber such that the liquid will flow up through the bed of green plant material and to the top of the chamber for delivery to a collection device. During or after delivery of the extract to the collection device, the SCF may be depressurized to below the desired pressure to collect the desired extract. In obtaining another embodiment of the invention, the extraction method is performed by counterflowing the SCF relative to the movement of the green plant material.

Importantly, the temperature and pressure may be controlled with conventional devices such as conventional pumps, valves, and/or heat exchangers before, during and/or after extraction to optimize the concentration or ratio of lutein, β-carotene, and fatty acids in a particular extraction. After leaving the extraction chamber, a pressure reduction valve may be positioned prior to the collection device intake to effect release or precipitation of the desired extract alone, or onto a specific carrier material in the collection device. A suitable double valve at the bottom of the collection device allows for periodic removal of the extract (with or without the carrier). The vented carbon dioxide liquid from the top of the collection device at a reduced pressure may then be recycled to a filter system and recompressed to high pressure for use in a second extraction function in the extraction vessel. Extraction is continued until an appropriate degree of desired product is isolated from the plant material being processed. The volume of SCF needed for the desired extraction depends on the pressure and temperature used for each product obtained. Typically 5-50 cubic feet of SCF are needed for each cubic foot of plant concentrate extracted. The ratio between the volume of SCF and green plant material may be referred to as the solvent-to-feed ratio, and may more particularly range from 10:1 to 50:1.

The supercritical extraction may performed under at least two different pressure and temperature conditions within the extraction chamber to vary the yield and/or ratio of lutein, β-carotene, and fatty acids in a particular extraction. For example, in one embodiment, at a first pressure and temperature, a first extract containing relatively more amount of β-carotene than lutein and fatty acids may be obtained, while at a second pressure and temperature, a second extract containing relatively more amount of lutein than β-carotene and fatty acids in the first extract may be obtained.

However, the second supercritical fluid extraction (or other additional extractions) does not necessarily have to be performed at both a different temperature and a different pressure than the first extraction. Rather, one or both of the temperature and pressure may be changed between extractions to achieve a desired result. Thus, as used herein, changes to the “pressure and temperature,” or to the “pressure and temperature conditions” refer to changes in the overall condition under which the extraction is performed, rather than to changes in both the temperature and pressure.

In this manner, an extract may be obtained having a controlled combination of lutein, β-carotene, and EFAs. This may be beneficial for certain applications, because it has been recognized that β-carotene and lutein are important in preserving eye health in that the lutein is concentrated in the macula, and β-carotene is converted to Vitamin A, which is critical to night vision and overall retinal health. Furthermore, EFAs might improve the absorption of the lutein and β-carotene. Thus, a blended, controlled mixture of β-carotene and lutein with a suitable concentration of EFAs is a good nutritional supplement for maintaining and/or improving eye health.

In one embodiment, the desired extract may contain about equal concentrations of lutein, β-carotene, and EFAs. For example, the extract may include concentrations of lutein, β-carotene, and EFAs between about 20-40 wt %, more particularly about 33 wt %.

Additionally, the supercritical extraction process described herein and used to obtain the present invention can be used to remove chlorophyll and other undesired materials, including flavor and odor-producing compounds, and hormones such as coumesterol. Thus, in one embodiment, at least one extract includes lutein, β-carotene, EFAs, and is substantially free of hormones such as coumesterol, odor and flavor producing compounds, and chlorophyll.

Although the pressure, temperature and volume at which the supercritical extractions are performed are related, each of these variables or conditions may be independently adjusted and/or optimized to produce one or more extracts having a specific concentration and/or ratio of lutein, β-carotene, and EFAs and/or other beneficial substituents. As an example, if a substantially high level of β-carotene is desired from alfalfa, an initial supercritical fluid extraction under low temperature and/or low pressure (e.g., 32° C.; 20 MPa; 20-50 volumes of CO₂) may be performed such that substantial portions of β-carotene will be isolated and concentrated in the extract. If higher temperatures and/or higher pressures (e.g., 43° C.; 50 MPa; 20-30 volumes) are used, the lutein and β-carotene may be more highly concentrated than the fatty acids in a single extract.

Furthermore, the volume of supercritical fluid needed to extract lutein, β-carotene, and EFAs may depend on the pressure and temperature at which the extraction is performed. For example, under low temperature and pressure conditions, it may be desirable to use a greater volume of SCF to obtain a particularly desired ratio in the extract. However, under higher temperature and pressure conditions, a lower volume of SCF may be required to obtain a different particularly desired ratio in the extract. In this manner, it is possible to adjust or optimize the extraction pressure, temperature, and/or volume to obtain extracts having a desired concentration, ratio, and/or purity of lutein, β-carotene, and EFAs. In certain embodiments, it may be desirable to perform at least a third extraction at a third temperature and/or pressure. For example, saponins may be isolated and extracted under higher pressure and/or temperature conditions than lutein and β-carotene are.

### Post-Extraction Processing

Optionally, after separation, the extract may be further processed to produce a desired end product. For example, a secondary column fractionation step may be used to further concentrate and purify the extract. Additionally, the first or second extract may be purified with simple non-toxic solvents such as food grade ethanol, a vegetable oil, or water to provide a substance that is crystalline and essentially pure and free of any potentially toxic chemicals, even on a trace level. Typically, lutein is concentrated to 5-50% concentration in oils or dry form for bulk markets. In one embodiment, the first and second extracts are combined before or after separation in order to provide an end product having a controlled concentration of lutein, β-carotene, and fatty acids. Advantageously, in embodiments that utilize multiple extractions to obtain a controlled, relative concentration of lutein, β-carotene, and fatty acids (and other nutrients), a post-extraction processing may be curtailed or completely eliminated.

The lutein, β-carotene, and EFAs in the extract may be also further processed by blending or milling with a suitable base material (e.g., green plant protein concentrate or other blending agents) to form an end product suitable for human consumption. This blending or milling step may take place in the collection device wherein the extract is precipitated into the base material. A suitable double valve at the bottom of the collection device may then be actuated to release the blended extract. In this manner, protein concentrates or blending agents may be used as an absorption agent in the lower pressure collection vessels.

### End Products

The extract may be combined with a suitable carrier to form an end product. The end product may be a powder, an agglomerated powder, a solution in edible oil, capsules, or microspheres that includes the extract. Alternatively, a protein matrix or beadlets may be produced to protect the extract from deterioration or oxidation. It may be analyzed for specific carotenoid content and then mixed with alfalfa or plant based natural fillers, sugars, gelatins, or starches to form a desired standardized dry product. In one embodiment, the extract is combined with a green chloroplast-rich fraction of alfalfa (which may also be used as the starting green plant material) such that an end product will contain only a single-source ingredient and may be labeled as 100% alfalfa based. The use of the green chloroplast fraction of alfalfa as the carrier in the final product is nutritionally beneficial because of the high content of useful proteins, vitamins, amino acids, chlorophyll, and other compounds in the fraction in addition to the presence of the concentrated lutein, β-carotene, and fatty acids. Furthermore, the end product is then derived from a single source plant product, without additional fillers or additives. The desired mixture of nutritional supplement will be set at 30% of B-Carotene, Lutein and Omega 3 and 6 Fatty acids with other minor fats. The final end products will be adjusted to about 5 wt % Lutein, about 5 wt % β-Carotene and about 5% wt essential fatty acids with alfalfa protein or with other fillers.

One example of a suitable carrier is cyclodextran. Cyclodextrans are 6 or 7 glucose units in cyclic form, known as Beta and Gamma cyclodextrans, respectively. These cyclodextrans contain an internal hydrophobic area while the external area of the molecules are very hydrophilic in nature. With these unique properties, these cyclodextrans are soluble in water, but are also able to protect the double bonds of lutein, B-carotene, and EFAs by internalizing these molecules. These properties make cyclodextrans a particularly suitable carrier for the xanthophylls, carotenes and fatty acids of the present invention. Nutritional supplements utilizing cyclodextrans as the carrier form stable blended products because one mole equivalent of cyclodextrans will entrain one mole of lutein or other oxygen sensitive molecules.

### EXAMPLES

Several tests with various times, various pressures, and additions of 3-4% ethanol resulted in different mixtures of fatty acids, B-Carotene and Lutein. The assays for the β-carotene and Lutein were done with Silica Gel Thin Layer Chromatography plates using a hexane:acetone solvent 70:30 mixture for plate development. The standards and the test material spots were removed from the plates into test tubes for assay. Addition of 2.0 ml of ethanol was used to elude the B-carotene and Lutein spots to measure the optical absorption at 457 nm to compare to the standard curves. In all cases of alfalfa protein concentrates at different times was typically 58% B-Carotene and 42% lutein with a variance of +/−12%.

Further embodiments of the invention are described below.

### Example 1

Fresh field chopped alfalfa was run through a hammermill to rupture plant cells. The tip speed of the hammers was set at 15,000 feet per minute to crush the green wet (80% moisture) material without causing the material to be pulped or broken into smaller pieces. The crushed material was run through a single (6 inch) screw press (Model Number VP6, available from Vincent Corp., Tampa, Fla.) such that the outlet restriction (set at 25 psi) produced high continuous pressure to effect separation of green plant juices from the plant fibers. The long barrel screw has a fine barrel screen to allow juice to flow from the fiber. The ratio of juice to fiber was about 1:1, however, the yield of juice to fiber will be less if the starting material is old or more matured, or if it is naturally dryer than lush pre-bloom growing alfalfa. The juice was immediately heated from ambient temperature with a double boiler system with a propane burner such that the juice was heated within 5-10 minutes after production to between 82-85° C. to cause heat coagulation of the green and white (cytoplasmic) proteins. The green protein coagulum was separated with a weir-type screen to separate the green “curd” from the brown, waste plant juices.

The green, wet protein “curd” (i.e., the green plant material) was immediately dried in a continuous perforated temperature controlled zone dryer such that limited heat (below 85° C.) with limited air at 5-10% relative humidity produced a dry granular material. The wet protein curd started at approximately 75% moisture and was dried to 8% moisture. This material was then extracted or stored in oxygen-excluding bags or containers in the dark at room temperature until extracted.

The green plant material was then transferred to a very high pressure extraction chamber (about 5 cm×50 cm) having round thick-walls, and being engineered to withstand pressures of up to about 70 MPa (10,150 psi). The chamber was brought up to pressure and temperature with 20 MPa carbon dioxide fluid at 30° C. The temperature and pressure of the SCF stream with an injected 3% liquid ethanol (vol./vol.) entrainer were regulated by a high-pressure, carbon-dioxide pump and heat exchanger controlled with water in a tube-and-shell system. This extraction continued until the β-carotene (about 27 bed volumes) was removed as measured in side port sampling at the top of the column outlet line. The pressure was then increased to 65 MPa to extract the lutein from the green plant material with about 20 bed volumes.

The desired extracts were collected after extraction into a small but tall (1 meter) tower with reduced pressure through reducing valves such that the lutein, β-carotene, and fatty acid fractions were collected into chambers with dried green protein powder at 10 MPa and 40 MPa respectively. The lower ¼ of the collection vessel has large valves to allow the desired fractions to fall out into the protein fractions such that after the separation of the fractions, the lower collection chamber was sealed off, and the pressure released to remove the end products.

The yield in this example was about 2.4 grams of lutein, 2.6 grams β-carotene, and 4 grams of fatty acids per kilogram of dry (6% moisture) starting material. The products were tested for purity without the blending with the green protein fraction with silica-gel HPLC columns and were 80% and 72% pure β-carotene and lutein, respectively. The drying of the green protein is critical in preserving the desired end products since the dried materials ranged from 0.6 to 3.4 grams of each carotenoid as measured with high performance liquid chromatography (HPLC) with known pure standards (available from Sigma Chemical, St. Louis, Mo.).

### Example 2

### Prophetic

In this prophetic example, a final product containing an essential extract is formed. The fresh field chopped alfalfa is treated in the same manner as in Example 1, except that the first supercritical extraction is performed at 15 MPa at about 80° F. for about 10-15 minutes and the second supercritical extraction is performed at 40 MPa at about 80° F. for about 60 minutes. minutes. The yield of essential extract in this example is about 33 wt % lutein, 33 wt % β-carotene, and 33 wt % fatty acids. The fatty acids are about 65 wt % linolenic acid, about 30 wt % linoleic acid and about 5 wt % other fatty acids. The products may be tested for purity without blending with the green protein fraction with silica-gel HPLC columns.

An aqueous slurry of cyclodextran is formed by mixing a dried powder form of commercially available cyclodextran with water to form an aqueous slurry of cyclodextran. The dried essential extract is then mixed with ethanol (Please provide concentration) to form a slurry of essential extract. The slurry of cyclodextran and the slurry of essential extract are then mixed to form a combined slurry. The combined slurry is then centrifuged. The solid material is removed from the centrifuge and then dried (please provide temperature and time of drying) to form the final product. The final product contains about between about 60-90 wt % cyclodextran and/or other starches, about 5% lutein, about 5% β-carotene, and about 5% fatty acid.

1. An extract derived from green plant materials comprising at least one xanthophyll, at least one hydrocarbon carotene, and at least one fatty acid, each having a weight percent of between about 20-40%.

2. The extract of claim 1 wherein extract includes substantially equal weight percents for the at least one xanthophyll, hydrocarbon carotene and fatty acid.

3. The extract of claim 1 wherein the green plant material includes dried green plants or fractions of green plants.

4. The extract of claim 1 wherein the green plant material is derived from alfalfa.

5. The extract of claim 1 wherein at least one xanthophyll is lutein.

6. The extract of claim 1 wherein at least one xanthophyll is selected from the group consisting of lutein, zeaxanthin, astaxanthin, canthaxanthin, capsorubin, cryptoxanthin, and combinations thereof.

7. The extract of claim 1 wherein at least one hydrocarbon carotene is β-carotene.

8. The extract of claim 1 wherein the at least one hydrocarbon carotene is selected from the group consisting of α-carotene, β-carotene, lycopene, and combinations thereof

9. The extract of claim 1 wherein the at least one fatty acid is a linolenic acid.

10. The extract of claim 1 wherein the at least one fatty acid is linoleic acid.

11. The extract of claim 1 wherein the at least one fatty acid is selected from the group consisting of a linolenic acid, a linoleic acid, a palmitic acid, an oleic acid, and combinations thereof.

12. The extract of claim 1 wherein the extract is substantially free of hormones.

13. The extract of claim 1 wherein the extract is substantially free of hormones.

14. The extract of claim 1 comprising at least two fatty acids, wherein at least a first fatty acid is an omega-3 essential fatty acid, and at least a second fatty acid is an omega-6 essential fatty acid.

15. The extract of claim 14 wherein the weight percent ratio of omega-3 essential fatty acid to the weight percent omega-6 essential fatty acid is not greater than about 5 to 1, respectively.

16. The extract of claim 14 wherein the weight percent ratio of omega-3 essential fatty acid to the weight percent ratio of omega-3 essential fatty acid to the weight percent omega-6 essential fatty acid is not greater than about 2 to 1, respectively.

17. A composition derived from an extract from green plant materials, the composition comprising about 60-90 weight percent carrier and at least about 5 weight percent of each of at least on xanthophyll, at least one hydrocarbon carotene, and at least one fatty acid.

18. The composition of claim 17 wherein the carrier includes a cylcodextran.

19. the composition of claim 17 comprising about equal weight percents of the at least one xanthophyll, at least one hydrocarbon carotene, and at least one fatty acid.

20. An extract formed by the process comprising:
performing a first supercritical fluid extraction of a green plant material at a first pressure and temperature to obtain a first extract;
performing at least one additional supercritical fluid extraction of the green plant material at least one additional pressure and temperature to obtain at least one additional extract, wherein at least one of the additional extracts includes a higher concentration of lutein than in the first extract; and
separating at least one of the additional extracts from the supercritical fluid.

21. The extract of claim 20 further comprising at least one carotene.

22. The extract of claim 20 further comprising at least one fatty acid.
